(19) United States
(12) Patent Application Publication
(10) Pub. No.: US 2024/0218461 A1
(43) Pub. Date: Jul. 4, 2024

(54) DNA BARCODE FOR SCREENING TOTAL SOLUBLE AMINO ACID CONTENT INDEX OF FLOCCULARIA LUTEOVIRENS

(71) Applicant: XIZANG VOCATIONAL TECHNICAL COLLEGE, LHASA (CN)

(72) Inventors: MANJUN YANG, LHASA (CN); CAIXIA ZHANG, LHASA (CN); YISHUI CAI, LHASA (CN)

(21) Appl. No.: 18/417,251

(22) Filed: Jan. 19, 2024

Related U.S. Application Data
(63) Continuation of application No. PCT/CN2022/109952, filed on Aug. 3, 2022.

(30) Foreign Application Priority Data
Nov. 19, 2021 (CN) 202111399046.1

(51) Int. Cl. C12Q 1/6895

(52) U.S. Cl. CPC C12Q 1/6895

(57) ABSTRACT

The present invention discloses a DNA barcode, a primer group and application for screening a total soluble amino acid content index of Floccularia luteovirens, belonging to the technical field of screening of edible fungi germplasm resources. Compared with a traditional breeding method and other existing DNA barcode technologies, the present invention has the advantages of time saving, labor saving, money saving, accuracy and high efficiency, plays a positive role in original place identification and genetic breeding of high-quality Floccularia luteovirens, and also provides an effective method for identification and protection of germplasm resources.

### SEQUENCE LISTING

The instant application contains a Sequence Listing which has been submitted electronically in ST.26 format and is hereby incorporated by reference in its entirety. Said ST.26 copy, created on Jan. 18, 2024, is named Sequence Listing.xml and is 14,110 bytes in size.

### TECHNICAL FIELD

The present invention relates to the technical field of screening of edible fungi germplasm resources, in particular to a DNA barcode, a primer group and application for screening a total soluble amino acid content index of Floccularia luteovirens.

### BACKGROUND

Floccularia luteovirens in a gold yellow color, also known as yellow mushroom and golden mushroom, is a high-quality edible fungus with a unique flavor, which cannot be cultivated artificially at present. Wild Floccularia luteovirens is mainly distributed in Qinghai-Tibet Plateau; and the main producing areas include Dangxiong County of Tibet Autonomous Region, Qilian County of Qinghai Province and Shiqu County of Sichuan Province, and the quality of these three main producing areas is the best. Main indexes to evaluate the nutritional value, flavor and biological activity of Floccularia luteovirens include: high contents and strong antioxidant activity of total soluble proteins, total soluble amino acids, total polyphenols, total polysaccharides and total fat. In different producing places, Floccularia luteovirens has different nutritional values, different flavors, different biological activities and different market prices. In the past, the breeding of Floccularia luteovirens was mainly determined by a morphological method combined with beneficial content indexes. However, due to the influence of the special climate environment of Qinghai-Tibet Plateau, the phenomena of different objects with the same name and the same object with different names often appeared to Floccularia luteovirens produced in different areas, so morphological identification was difficult to realize effective distinguishment. More difficultly, it is impossible to screen out high-quality strains with high contents of total soluble proteins, total soluble amino acids, total polyphenols, total polysaccharides and total fat, and strong antioxidant activity by the morphological method. In addition, due to high altitude of main producing areas thereof, it is also difficult to collect samples.

A DNA barcode molecular identification technology is a molecular biology technology based on DNA barcodes (conserved and stable genetic DNA sequences in a genome) to recognize and identify species and excellent quality. It is the effective supplement and expansion of traditional breeding methods, and can accurately and effectively identify samples when samples are incomplete in morphologies or lack morphological structures (processed products such as powder, etc.). In order to realize effective development and utilization of Floccularia luteovirens, it is particularly important and urgent to screen different producing areas of Floccularia luteovirens strains with assistance of the DNA barcode molecular identification technology. In the existing DNA barcode technology, non-coding regions or conserved gene sequences in ITS (internal transcribed spacer in ribosomal RNA) and mitochondria are mainly used for species object identification; the operation of restriction fragment length polymorphism (RFLP) is very complicated; reliability and repeatability of results are poor; random amplified polymorphic DNA (RAPD) is easily disturbed, which requires a high technical level of operators and is difficult to popularize in assisted breeding; and single nucleotidepolymorphism (SNP) has high requirements for equipment and high cost.

Therefore, in view of shortcomings of traditional breeding methods that the breeding of Floccularia luteovirens strains is not accurate enough, time-consuming and labor-consuming, an urgent problem to be solved by those skilled in the art is how to provide a DNA barcode that can accurately and quickly identify the strains of Floccularia luteovirens and realize high-quality breeding, which has the characteristics of low cost, high efficiency, simple operation, stable and reliable results and good repeatability.

### SUMMARY

In view of this, the present invention provides a DNA barcode and a primer group for screening a total soluble amino acid content index of Floccularia luteovirens, which can quickly and accurately screen out strains with a high total soluble amino acid content of Floccularia luteovirens, and provides a favorable auxiliary means for breeding high-quality Floccularia luteovirens.

In order to achieve the above purpose, the present invention adopts the following technical solution:

A DNA barcode for screening a total soluble amino acid content index of Floccularia luteovirens, wherein a nucleotide sequence of the DNA barcode includes one or more of:
SEQ ID NO:3,
and/or SEQ ID NO:4,
and/or a combination of SEQ ID NO:3 and SEQ ID NO:4,
and/or SEQ ID NO:7,
and/or SEQ ID NO:8,
and/or a combination of SEQ ID NO:7 and SEQ ID NO:8,
and/or a combination of SEQ ID NO: 11 and SEQ ID NO:12,
and/or SEQ ID NO:12,
and/or a combination of SEQ ID NO: 12 and SEQ ID NO:13.

According to the present invention, based on all simple sequence repeats (SSR) in the whole genome of Floccularia luteovirens, fluorescent PCR amplification is carried out; and a DNA barcode which effectively corresponds to a total soluble amino acid content is established. Compared with the DNA barcode of the present invention, the fragment obtained by amplification can quickly and accurately screen out strains with a high total soluble amino acid content of Floccularia luteovirens, which provides favorable assistance for breeding of Floccularia luteovirens.

Another purpose of the present invention is to provide a primer group for amplifying the DNA barcode for screening the total soluble amino acid content index of Floccularia luteovirens, wherein a nucleotide sequence of the primer group includes one or more groups of:
SEQ ID NO: 1 and SEQ ID NO:2,
and/or SEQ ID NO:5 and SEQ ID NO:6,
and/or SEQ ID NO:9 and SEQ ID NO:10.

As a preferred technical solution of the present invention, the nucleotide sequence of the primer group includes:
such as SEQ ID NO: 1 and SEQ ID NO:2,
and SEQ ID NO:5 and SEQ ID NO:6,
and SEQ ID NO:9 and SEQ ID NO:10.

Different primer groups of the present invention can be used alone or in combination to screen the total soluble amino acid content of Floccularia luteovirens; and when all primer groups are used together, the screening accuracy is the highest.

Another purpose of the present invention is to provide a method for screening Floccularia luteovirens by using the total soluble amino acid content index, which comprises the following steps:
S1, extracting genomic DNA of a sample to be tested;
S2, taking the genomic DNA in S1 as a template, and performing a fluorescent PCR amplification reaction on one or more groups of the above primers to obtain an amplification product;
S3, detecting the amplification product of S2 by capillary fluorescence electrophoresis, and judging by the number of fragments, the number of SSR loci, SSR repetitive elements and repeating times of the amplification product.

As a preferred technical solution of the present invention, a judgment standard in step S3 is:
when the primer group of SEQ ID NO:1 and SEQ ID NO:2 is amplified to obtain a 277 bp fragment containing 5 TGG repetitive elements and a 280 bp fragment containing 6 TGG repetitive elements;
and/or the primer group of SEQ ID NO:5 and SEQ ID NO:6 is amplified to obtain a 219 bp fragment containing 6 TA repetitive elements;
and/or the primer group of SEQ ID NO:9 and SEQ ID NO: 10 is amplified to obtain a 239 bp fragment containing 6 TCA repetitive elements and a 242 bp fragment containing 7 TCA repetitive elements, the Floccularia luteovirens is determined as Floccularia luteovirens with high total soluble amino acid content.

As a preferred technical solution of the present invention, a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

Further preferably, the concentrations of the forward primer, the reverse primer and the M13 primer with fluorescence are all 10 uM.

As a preferred technical solution of the present invention, a fluorescent PCR amplification reaction procedure in step S2 is:
performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

Further another purpose of the present invention is to provide application of the DNA barcode and/or the primer group in preparation of a product for screening high-quality Floccularia luteovirens with the total soluble amino acid content index.

Further another purpose of the present invention is to provide a product for screening high-quality Floccularia luteovirens with the total soluble amino acid content index, which comprises one or more of the above primer groups, and satisfies the standard that the primer group of SEQ ID NO:1 and SEQ ID NO:2 is amplified to obtain a 277 bp fragment containing 5 TGG repetitive elements and a 280 bp fragment containing 6 TGG repetitive elements;
and/or the primer group of SEQ ID NO:5 and SEQ ID NO:6 is amplified to obtain a 219 bp fragment containing 6 TA repetitive elements;
and/or the primer group of SEQ ID NO:9 and SEQ ID NO: 10 is amplified to obtain a 239 bp fragment containing 6 TCA repetitive elements and a 242 bp fragment containing 7 TCA repetitive elements.

As a preferred technical solution of the present invention, the product is a kit.

According to the above technical solution, compared with the prior art, the present invention discloses and provides the DNA barcode and the primer group for screening the total soluble amino acid content index, which can utilize wild samples of Floccularia luteovirens and a small amount of tissues or hyphae to carry out character breeding of high-quality strains; identification can be carried out at different growth stages such as mycelium, primordia, fruiting bodies and spores; and the present invention has the short detection period, simple operation, no waste, stable and reliable results and good repeatability, and overcomes the shortcomings of traditional breeding methods that the breeding of Floccularia luteovirens strains is not accurate enough, time-consuming and labor-consuming.

Compared with a traditional breeding method and other existing DNA barcode technologies, the present invention has the advantages of time saving, labor saving, money saving, accuracy and high efficiency, plays a positive role in character screening and genetic breeding of high-quality Floccularia luteovirens, and also provides an effective method for identification and protection of germplasm resources.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a diagram of comparison results of total soluble amino acid contents of test example, reference example 1 and reference example 2 according to the present invention;

FIG. 2A is a diagram of reference example 1 of fluorescent PCR amplification using primer 1 according to the present invention;

FIG. 2B is a diagram of reference example 2 of fluorescent PCR amplification using primer 1 according to the present invention;

FIG. 2C is a diagram of test example of fluorescent PCR amplification using primer 1 according to the present invention;

FIG. 3A is a diagram of reference example 1 of fluorescent PCR amplification using primer 2 according to the present invention;

FIG. 3B is a diagram of reference example 2 of fluorescent PCR amplification using primer 2 according to the present invention;

FIG. 3C is a diagram of test example of fluorescent PCR amplification using primer 2 according to the present invention;

FIG. 4A is a diagram of reference example 1 of fluorescent PCR amplification using primer 3 according to the present invention;

FIG. 4B is a diagram of reference example 2 of fluorescent PCR amplification using primer 3 according to the present invention; and

FIG. 4C is a diagram of test example of fluorescent PCR amplification using primer 3 according to the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a DNA barcode, a primer group and application for screening a total soluble amino acid content index of Floccularia luteovirens. Reagents used are commercially available; and sources thereof are not specifically limited. Unless otherwise mentioned, test methods used are conventional methods.

### Embodiment 1 Establishment of a DNA Barcode of Floccularia luteovirens

Samples of Floccularia luteovirens are collected from Dangxiong County of Tibet Autonomous Region, Qilian County of Qinghai Province and Shiqu County of Sichuan Province and are treated by genome sequencing; and SSR loci in the genome sequences are analyzed by MISA program.

Primers are designed for PCR amplification of the SSR loci; the primers that can amplify the corresponding fragments are reserved; and invalid primers are discarded.

The total soluble amino acid contents of Floccularia luteovirens samples from Dangxiong County of Tibet Autonomous Region, Qilian County of Qinghai Province and Shiqu County of Sichuan Province are determined.

The samples from the above three producing areas are amplified by effective primers and detected by capillary electrophoresis. The simple sequence repeat (SSR) locus corresponding to the total soluble amino acid content is established by analysis. Finally, three pairs of primers (see Table 1) are obtained. The three pairs of primers are used to amplify the sample genome; and the fragment polymorphism obtained can assist in screening Floccularia luteovirens with the high total soluble amino acid content.

TABLE 1
Specific primers for screening of high-quality strains of Floccularia
luteovirens with high total soluble amino acid contents

| | | | | Denaturation | |
|---|---|---|---|---|---|
| | | Denaturation | | temperature | SSR |
| No. | Forward primer | temperature (° C.) | Reverse primer | (° C.) | element |
| 1 | TGTAAAACGACGGC | 60.038 | GTGCGTCTCA | 59.968 | TGG |
| | CAGTTGACGTTGGTG | | GTCCAGTTGA, | | |
| | GAGATTCCG, as | | as shown in | | |
| | shown in SEQ ID No: 1 | | SEQ ID No: 2 | | |
| 2 | TGTAAAACGACGGC | 59.968 | CGATGTCTTA | 60.248 | TA |
| | CAGTGTGACACAGC | | CGGCAGCGT | | |
| | AACTCGGAGA, as | | A, as shown in | | |
| | shown in SEQ ID No: 5 | | SEQ ID No: 6 | | |
| 3 | TGTAAAACGACGGC | 60.038 | ACCACGTCA | 60.037 | TCA |
| | CAGTCGGTCGAGCTT | | GCCAGAAGA | | |
| | CAGGAGTTT, as shown | | TG, as shown in | | |
| | in SEQ ID No: 9 | | SEQ ID No: 10 | | |

### Embodiment 2 Amplification of SSR Specific Primers for Total Soluble Amino Acid Contents of Floccularia luteovirens Strains

(1) Extraction of total soluble amino acids

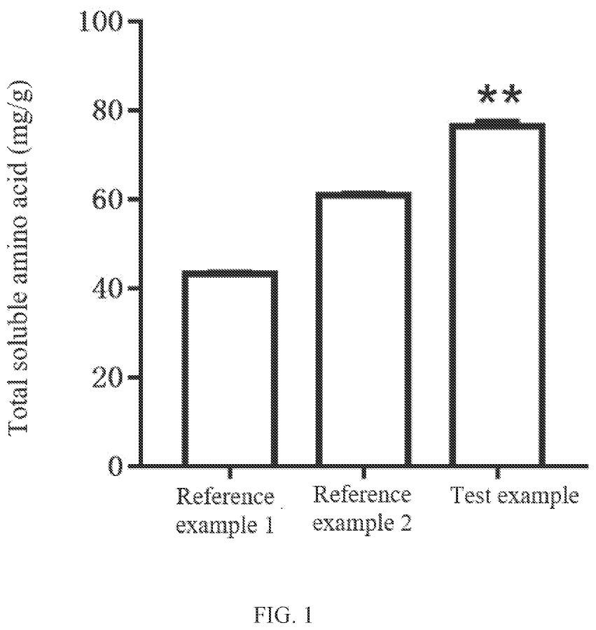

Fruiting bodies of Floccularia luteovirens are collected from Dangxiong County of Tibet Autonomous Region, Qilian County of Qinghai Province and Shiqu County of Sichuan Province, dehydrated by a vacuum freeze-drying method, smashed and sieved with a 50 mesh sieve; 1 g of dry powder is added with 10 mL of double distilled water, extracted with assistance of the 300 W ultrasonic wave for 30 min, and then centrifuged at 5000 r/min for 30 min; and the supernatant is taken to prepare a total soluble amino acid extracting solution. A total soluble amino acid content is determined by ninhydrin colorimetry. See the methods of Liu Changjiao et al. (Liu Changjiao, Yang Yueyue, Wang Ni and Yu Ping. Optimization of conditions for determining an amino acid content in okra by ninhydrin colorimetry [J]. China Food Additives, 2018, 1:187-193.). The content is converted into mg/g. The total soluble amino acid content of Floccularia luteovirens from Qilian County of Qinghai Province is 76.46 (+1.21) mg/g, which is determined as the test example. The total soluble amino acid content of Floccularia luteovirens from Dangxiong County of Tibet Autonomous Region is 43.18 (±0.58) mg/g, which is determined as reference example 1. The total soluble amino acid content of Floccularia luteovirens from Shiqu County of Sichuan Province is 61.03 (±0.51) mg/g, which is determined as reference example 2 (see FIG. 1).
(2) The genome of Floccularia luteovirens samples is extracted by an Ezup column fungus genomic DNA extraction kit (No. B518259) of Sangon Biotech (Shanghai) Co., Ltd., and diluted to 20 ng/μL for fluorescent PCR amplification.
(3) Fluorescent PCR amplification of the SSR DNA barcode is carried out with primers in Table 1.

Fluorescent PCR amplification reaction system (10 μL): 5 μL of 2×Taq PCR MasterMix, 1 μL of template (genomic DNA), 0.1 μL of forward primer, 0.4 μL of reverse primer (concentrations of the forward primer and the reverse primer are both 10 uM), and 0.4 μL of M13 primer with fluorescence (concentration of 10 uM), wherein a volume is fixed to 10 μL with sterile deionized water.

Reaction conditions: performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.
(4) After quantitative dilution of the PCR product, 1 μL of a PCR diluted products is denatured with 9 μL of formamide (including 1% internal standard), and then subjected to capillary fluorescence electrophoresis detection by a DNA sequencer ABI 3730×1. The internal standard, which is a molecular weight internal standard (also known as internal lane standard) LIZ-500 bp, is composed of 16 double-stranded DNA fragments labeled with LIZ fluorescein (orange), with molecular weights of 35, 50, 75, 100, 139, 150, 160, 200, 250, 300, 340, 350, 400, 450, 490 and 500 bp respectively. The fragment size in the amplification result electropherogram is equal to the actual bp number of the amplified fragment plus the M13 fluorescent primer (about 18 bp, with error of 1-2 bp). The amplified capillary electrophoresis peak is combined with the sequencing result; and the peak number indicates the number of heterozygous amplified fragments of the gene.
(5) The above methods are used to identify Floccularia luteovirens in test example, reference example 1 and reference example 2.

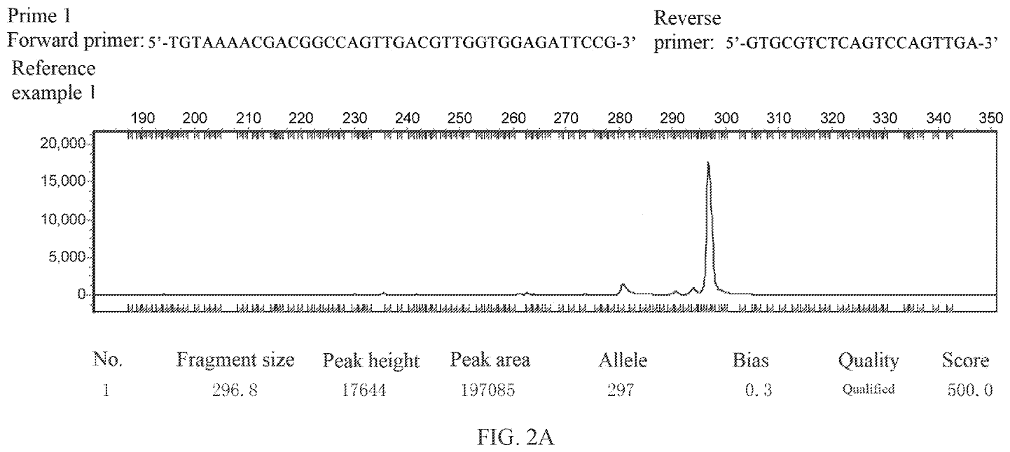

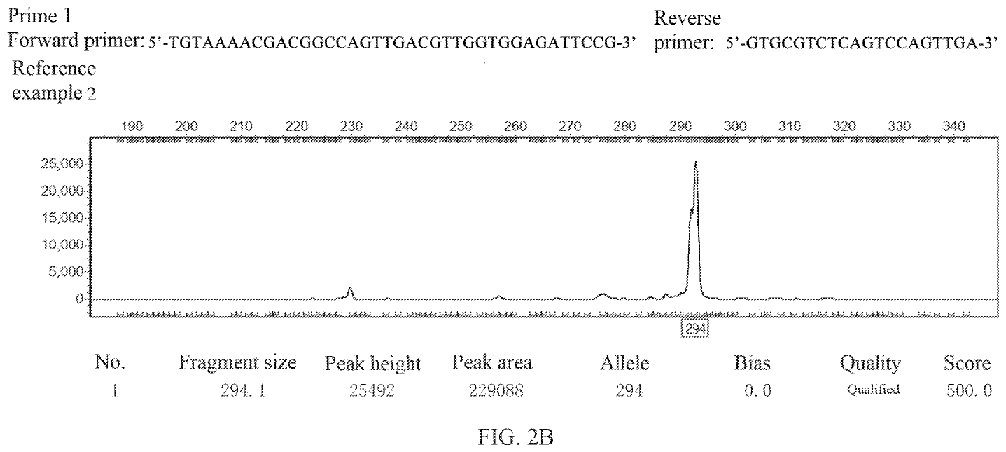

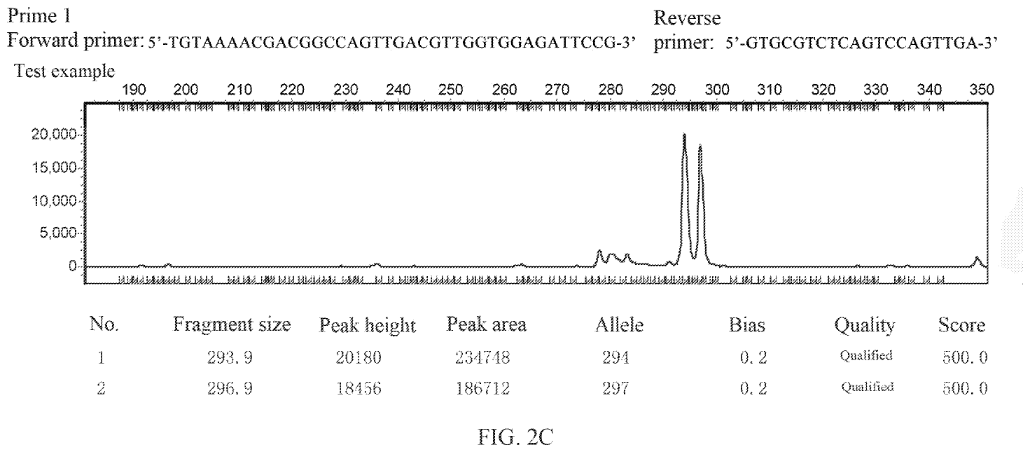

Amplification results of primer 1 are shown in FIGS. 2A-2C. When primer 1 is used for fluorescent PCR amplification, two fragments (two peaks) are obtained through amplification, which contain two SSR loci; and the SSR repetitive element is TGG. The amplified fragment obtained in the test example is characterized by containing a 277 bp fragment with 5 repeats and a 280 bp fragment with 6 repeats.

Amplified fragments of primer 1: (the statistical fragment length of electropherogram includes the M13 fluorescent primer; the specific sequence shows that the M13 fluorescent primer sequence (17 bp) is removed, with an error of 1 bp; and the underlined part is an SSR repetitive element.)

### 277 bp Amplified Fragment Sequence:

TGACGTTGGTGGAGATTCCGAAGATATTGGCGCTCTTGTAAAATTTGCTG
ATGATGAGACTCATGGTGTTGCGGCTGTTGCTGTTGCTGCAAATGAAGTT
GGTGGTGGTGGTGGTACAACTGCTCATCCTGTAACATCTGTTCGTGTTGA
CGCTGGCGAGTCTGCATCTGTCGTTGTAGCTGCTCTTCTTCAAAACGAGA
ACGGGGAGAAGAAAGAGGGAGAATATAAGGGAGGGAGACGGGCGCTGAGA
AAATGAATCAACTGGACTGAGACGCAC (as shown in SEQ ID
NO: 3)

### 280 bp Amplified Fragment Sequence:

TGACGTTGGTGGAGATTCCGAAGATATTGGCGCTCTTGTAAAATTTGCTG
ATGATGAGACTCATGGTGTTGCGGCTGTTGCTGTTGCTGCAAATGAAGTT
GGTGGTGGTGGTGGTGGTACAACTGCTCATCCTGTAACATCTGTTCGTGT
TGACGCTGGCGAGTCTGCATCTGTCGTTGTAGCTGCTCTTCTTCAAAACG
AGAACGGGGAGAAGAAAGAGGGAGAATATAAGGGAGGGAGACGGGCGCTG
AGAAAATGAATCAACTGGACTGAGACGCAC (as shown in SEQ ID
NO: 4).

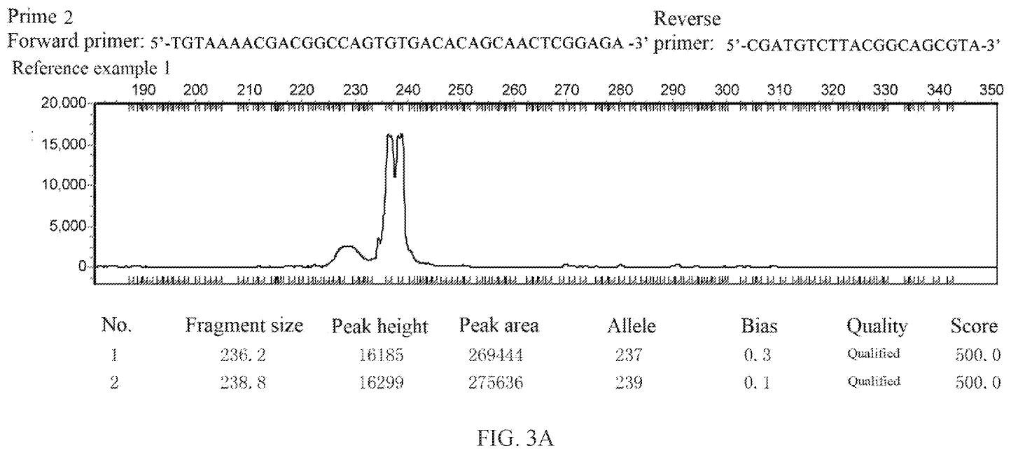

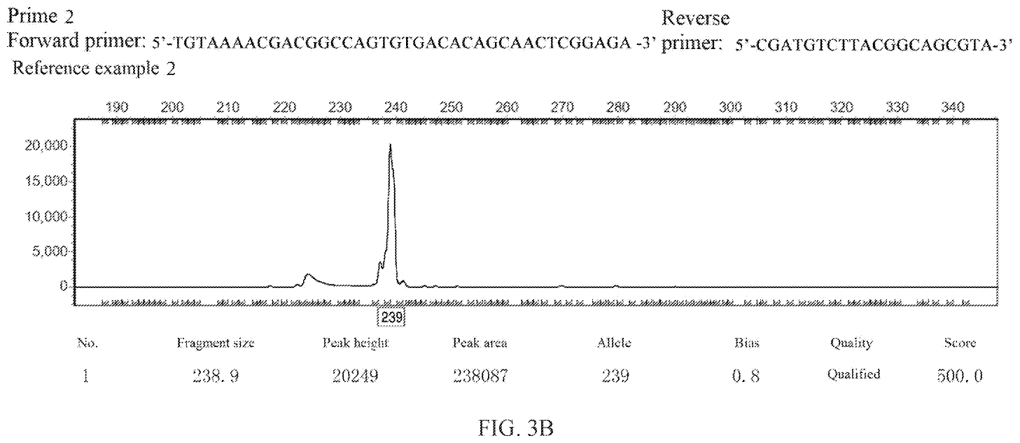

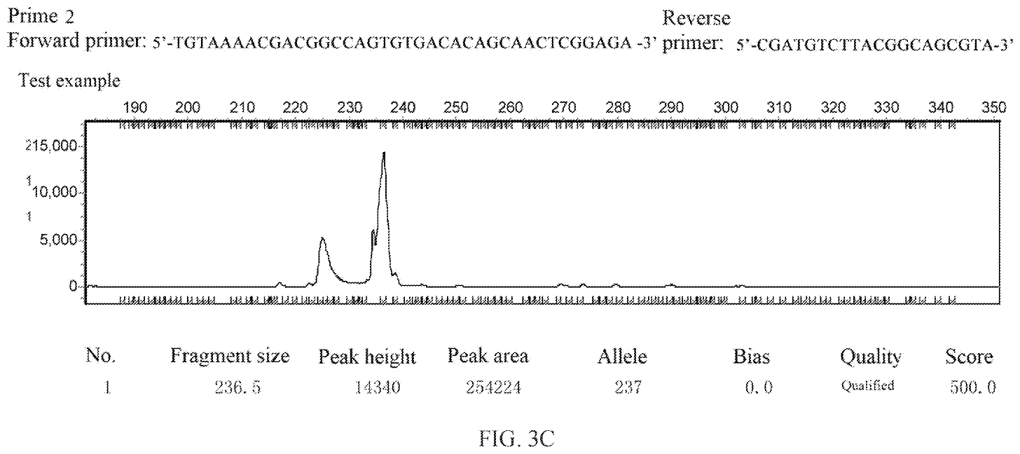

Amplification results of primer 2 are shown in FIGS. 3A-3C. When primer 2 is used for fluorescent PCR amplification, two fragments (two peaks) are obtained, which contain two SSR loci; and the SSR repetitive element is TA. The amplified fragment obtained in the test example is characterized by containing a 219 bp fragment with 6 repeats. In FIGS. 3A-3C, the amplified fragment of 225 bp belongs to nonspecific amplification and does not contain an SSR repetitive element.

Amplified fragments of primer 2: (the statistical fragment length of electropherogram includes the M13 fluorescent primer; the specific sequence shows that the M13 fluorescent primer sequence (18 bp) is removed; and the underlined part is an SSR repetitive element.)

### 219 bp Amplified Fragment Sequence:

GTGACACAGCAACTCGGAGAACTGTTGTACAAGTAAATGTTACACGAAGC
ACAACAGGAGAACTATATATATATAATTATGGACTGACGTGAAAATGCAG
GCGATTACTAGTCCCGTTAAGAGGTTGTTATGAAAGGAGGTAAGGAATAA
CTTGGAGAAAACGGAAGGTCCATCCCGACCAGAGTGCACGCATCAATAAT
ACGCTGCCGTAAGACATCG (as shown in SEQ ID NO: 7)

### 221 bp Amplified Fragment Sequence:

GTGACACAGCAACTCGGAGAACTGTTGTACAAGTAAATGTTACACGAAGC
ACAACAGGAGAACTATATATATATATAATTATGGACTGACGTGAAAATGC
AGGCGATTACTAGTCCCGTTAAGAGGTTGTTATGAAAGGAGGTAAGGAAT
AACTTGGAGAAAACGGAAGGTCCATCCCGACCAGAGTGCACGCATCAATA
ATACGCTGCCGTAAGACATCG (as shown in SEQ ID NO: 8)

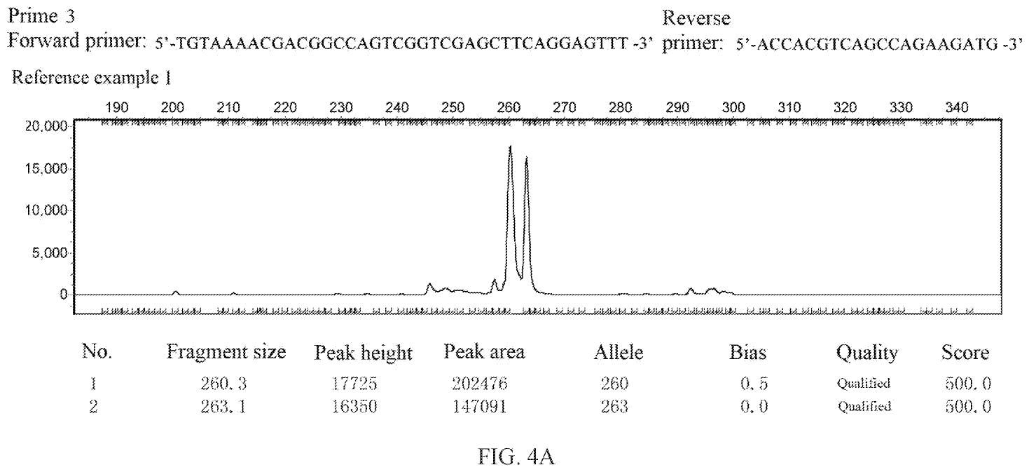

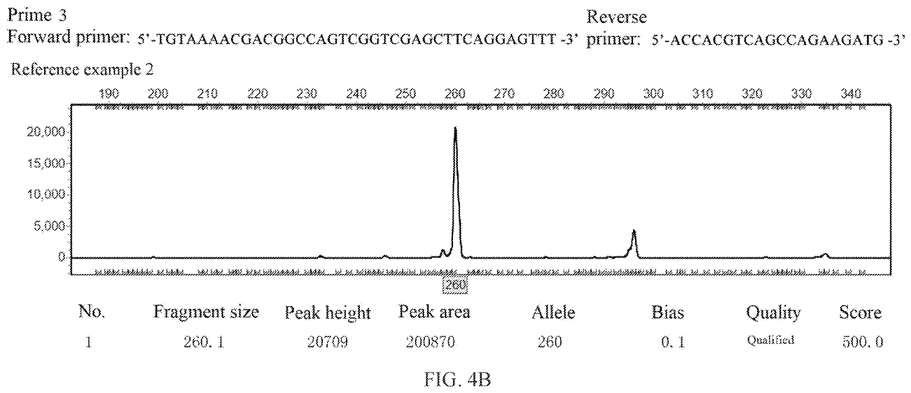

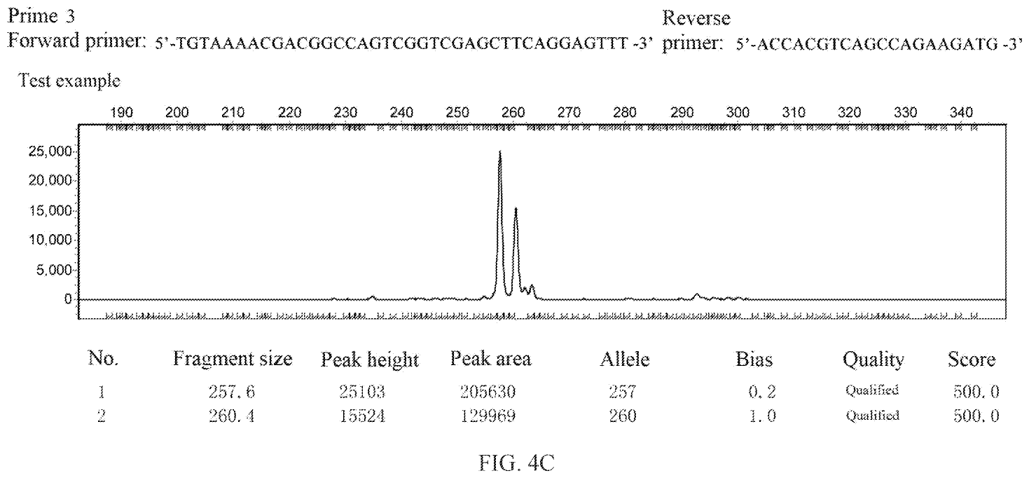

Amplification results of primer 3 are shown in FIGS. 4A-4C. When primer 3 is used for fluorescent PCR amplification, three fragments (three peaks) are obtained, which contain three SSR loci; and the SSR repetitive element is TCA. The amplified fragment obtained in the test example is characterized by containing a 239 bp fragment with 6 repeats and a 242 bp fragment with 7 repeats.

Amplified fragments of primer 3: (the statistical fragment length of electropherogram includes the M13 fluorescent primer; the specific sequence shows that the M13 fluorescent primer sequence (18 bp) is removed; and the underlined part is an SSR repetitive element.)

### 239 bp Amplified Fragment Sequence:

CGGTCGAGCTTCAGGAGTTTTGGGTTCGACGACCTCTTCTTCTTCCACAA
CATCAGTCTTGGATTCCACATCATTGTGCAGGAAGGATCGTTCCTTCTTC
CTCTTCTACGCTTCCTTCCTCAGAATAATTGGTGTCCTCGTCTTCATCAT
CATCATCATCATCGAGAATGACAGAATGACGACCTTTAAATGAGTAACCA
TCGAATACGTCAACGGTATCATCTTCTGGCTGACGTGGT (as shown
in SEQ ID NO: 11)

### 242 bp Amplified Fragment Sequence:

CGGTCGAGCTTCAGGAGTTTTGGGTTCGACGACCTCTTCTTCTTCCACAA
CATCAGTCTTGGATTCCACATCATTGTGCAGGAAGGATCGTTCCTTCTTC
CTCTTCTACGCTTCCTTCCTCAGAATAATTGGTGTCCTCGTCTTCATCAT
CATCATCATCATCATCGAGAATGACAGAATGACGACCTTTAAATGAGTAA
CCATCGAATACGTCAACGGTATCATCTTCTGGCTGACGTGGT (as
shown in SEQ ID NO: 12)

245 bp Amplified Fragment Sequence:

CGGTCGAGCTTCAGGAGTTTTGGGTTCGACGACCTCTTCTTCTTCCACAA
CATCAGTCTTGGATTCCACATCATTGTGCAGGAAGGATCGTTCCTTCTTC
CTCTTCTACGCTTCCTTCCTCAGAATAATTGGTGTCCTCGTCTTCATCAT
CATCATCATCATCATCATCGAGAATGACAGAATGACGACCTTTAAATGAG
TAACCATCGAATACGTCAACGGTATCATCTTCTGGCTGACGTGGT (as
shown in SEQ ID NO: 13)

Through comprehensive analysis of maps and sequencing results of test example, reference example 1 and reference example 2, the characteristic information of the DNA barcode of Floccularia luteovirens with the high total soluble amino acid content is shown in Table 2. Primer 1 carries out amplification to obtain the 277 bp fragment containing 5 TGG repetitive elements (as shown in SEQ ID NO:3) and the 280 bp fragment containing 6 TGG repetitive elements (as shown in SEQ ID NO:4); primer 2 carries out amplification to obtain the 219 bp fragment containing 6 TA repetitive elements (as shown in SEQ ID NO:7); and primer 3 carries out amplification to obtain a 239 bp fragment containing 6 TCA repetitive elements (as shown in SEQ ID NO:11) and the 242 bp fragment containing 7 TCA repetitive elements (as shown in SEQ ID NO: 12). Primers 1, 2 and 3 or any combination of the primers can be used for comprehensive detection and judgment. When primers 1, 2 and 3 are used together, the accuracy of screening the total soluble amino acid content index of Floccularia luteovirens is the best.

TABLE 2
DNA barcode characteristics of Floccularia luteovirens with high
total soluble amino acid content

| Total soluble | Primer | Primer | Primer |
|---|---|---|---|
| amino | 1 amplified | 2 amplified | 3 amplified |
| acid content | fragment (SSR) | fragment (SSR) | fragment (SSR) |
| Reference | 280 bp (6TGG) | 219 bp (6TA) | 242 bp (7TCA) |
| example 1 | | 221 bp (7TA) | 245 bp (8TCA) |
| Low content | | | |
| Reference | 277 bp (5TGG) | 221 bp (7TA) | 242 bp (7TCA) |
| example 2 | | | |
| Low content | | | |
| Test example | 277 bp (5TGG) | 219 bp (6TA) | 239 bp (6TCA) |
| High content | 280 bp (6TGG) | | 242 bp (7TCA) |

### Embodiment 3 Screening and Verification of a Total Soluble Amino Acid Content Index of Floccularia luteovirens

A DNA barcode of a total soluble amino acid content of Floccularia luteovirens is verified by blind testing.
Step 1, blind testing: taking samples from Qilian County of Qinghai Province with a total soluble amino acid content higher than or equal to 76.46 mg/g as an test group, taking samples from Dangxiong County of Tibet Autonomous Region and Shiqu County of Sichuan Province with total soluble amino acid contents lower than 76.46 mg/g (significance P<0.05) as a reference group 1 and a reference group 2, and taking 16 samples respectively, namely 48 samples in total, for blind testing;
step 2, testing: using primers (SEQ ID NO:1 and SEQ ID NO:2, SEQ ID NO:5 and SEQ ID NO:6, and SEQ ID NO:9 and SEQ ID NO:10) for amplification and capillary electrophoresis. One or more pairs of primer groups can be combined for amplification, and the blind testing samples can be distinguished by DNA barcode characteristics of the total soluble amino acid content;
step 3, unblinding: results are shown in Table 3. The unblinding results of 16 samples with high and low total soluble amino acid contents are all correct according to distinguishment by the DNA barcode characteristics of the total soluble amino acid content, indicating that the DNA barcode of the total soluble amino acid content is suitable for screening of the total soluble amino acid content character.

TABLE 3
Unblinding identification results based on DNA barcode characteristics of total
soluble amino acid content

| | Primer 1 | Primer 2 | Primer 3 | | |
|---|---|---|---|---|---|
| | amplified | amplified | amplified | | |
| Blind testing | fragment | fragment | fragment | Unblinding | Distinguishment |
| No. | (SSR) | (SSR) | (SSR) | No. | results |
| 2, 4, | 280 bp | 219 bp (6TA) | 242 bp | 33-48 | Total soluble |
| 5, 7, | (6TGG) | 221 bp (7TA) | (7TCA) | | amino acid |
| 8, 12, 14, 15, | | | 245 bp | | content of |
| 19, 22, 25, 26, | | | (8TCA) | | reference group |
| 34, 35, | | | | | 1 is low |
| 43,45 | | | | | |
| 1, 6, | 277 bp | 221 bp (7TA) | 242 bp | 17-32 | Total soluble |
| 9, 13, 16, 17, | (5TGG) | | (7TCA) | | amino acid |
| 20, 24, 28, 30, | | | | | content of |
| 33, 38, 39, 42, | | | | | reference group |
| 46, 47 | | | | | 2 is low |
| 3, 10, 11, 18, | 277 bp | 219 bp (6TA) | 239 bp | 1-16 | Total soluble |
| 21, 23, 27, 29, | (5TGG) | | (6TCA) | | amino acid |
| 31, 32, 36, 37, | 280 bp | | 242 bp | | content of test |
| 40, 41, 44, 48 | (6TGG) | | (7TCA) | | group is high |

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A DNA barcode for screening a total soluble amino acid content index of Floccularia luteovirens, wherein a nucleotide sequence of the DNA barcode comprises one or more of:
SEQ ID NO:3,
and/or SEQ ID NO:4,
and/or a combination of SEQ ID NO:3 and SEQ ID NO:4,
and/or SEQ ID NO:7,
and/or SEQ ID NO:8,
and/or a combination of SEQ ID NO:7 and SEQ ID NO:8,
and/or a combination of SEQ ID NO:11 and SEQ ID NO:12,
and/or SEQ ID NO:12,
and/or a combination of SEQ ID NO: 12 and SEQ ID NO:13.

2. A primer group for amplifying the DNA barcode for screening the total soluble amino acid content index of Floccularia luteovirens according to claim 1, wherein a nucleotide sequence of the primer group comprises one or more groups of:
SEQ ID NO: 1 and SEQ ID NO:2,
and/or SEQ ID NO:5 and SEQ ID NO:6,
and/or SEQ ID NO:9 and SEQ ID NO:10.

3. The primer group according to claim 2, wherein a nucleotide sequence of the primer group comprises:
such as SEQ ID NO:1 and SEQ ID NO:2,
and SEQ ID NO:5 and SEQ ID NO:6,
and SEQ ID NO:9 and SEQ ID NO:10.

4. A method for screening Floccularia luteovirens by using a total soluble amino acid content index, comprising the following steps:
S1, extracting genomic DNA of a sample to be tested;
S2, taking the genomic DNA in S1 as a template, and performing a fluorescent PCR amplification reaction on one or more groups of primers according to claim 2 to obtain an amplification product;
S3, detecting the amplification product of S2 by capillary fluorescence electrophoresis, and judging by the number of fragments, the number of SSR loci, SSR repetitive elements and repeating times of the amplification product.

5. The method for screening Floccularia luteovirens by using the total soluble amino acid content index according to claim 4, wherein a judgment standard in step S3 is:
when the primer group of SEQ ID NO:1 and SEQ ID NO:2 is amplified to obtain a 277 bp fragment containing 5 TGG repetitive elements and a 280 bp fragment containing 6 TGG repetitive elements;
and/or the primer group of SEQ ID NO:5 and SEQ ID NO:6 is amplified to obtain a 219 bp fragment containing 6 TA repetitive elements;
and/or the primer group of SEQ ID NO:9 and SEQ ID NO:10 is amplified to obtain a 239 bp fragment containing 6 TCA repetitive elements and a 242 bp fragment containing 7 TCA repetitive elements, the Floccularia luteovirens is determined as Floccularia luteovirens with high total soluble amino acid content.

6. The method for screening Floccularia luteovirens by using the total soluble amino acid content index according to claim 4, wherein a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

7. The method for screening Floccularia luteovirens by using the total soluble amino acid content index according to claim 6, wherein the concentrations of the forward primer, the reverse primer and the M13 primer with fluorescence are all 10 uM.

8. The method for screening Floccularia luteovirens by using the total soluble amino acid content index according to claim 4, wherein a fluorescent PCR amplification reaction procedure in step S2 is:
performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

9. An application of the DNA barcode according to claim 1 in preparation of a product for screening Floccularia luteovirens with the total soluble amino acid content index.

10. An application of the primer group according to claim 2 in preparation of a product for screening Floccularia luteovirens with the total soluble amino acid content index.

11. A product for screening high-quality Floccularia luteovirens with a total soluble amino acid content index, comprising one or more groups of the primer groups according to claim 2, and satisfying the standard that the primer group of SEQ ID NO:1 and SEQ ID NO:2 is amplified to obtain a 277 bp fragment containing 5 TGG repetitive elements and a 280 bp fragment containing 6 TGG repetitive elements;
and/or the primer group of SEQ ID NO:5 and SEQ ID NO:6 is amplified to obtain a 219 bp fragment containing 6 TA repetitive elements;
and/or the primer group of SEQ ID NO:9 and SEQ ID NO:10 is amplified to obtain a 239 bp fragment containing 6 TCA repetitive elements and a 242 bp fragment containing 7 TCA repetitive elements.


SEQUENCE LISTING (13 sequences)

SEQ ID NO: 1 - DNA, 38 bp, synthetic construct
  source 1..38: mol_type = other DNA
tgtaaaacgacggccagttgacgttggtggagattccg

SEQ ID NO: 2 - DNA, 20 bp, synthetic construct
  source 1..20: mol_type = other DNA
gtgcgtctcagtccagttga

SEQ ID NO: 3 - DNA, 277 bp, synthetic construct
  source 1..277: mol_type = other DNA
tgacgttggtggagattccgaagatattggcgctcttgtaaaatttgctgatgatgagactcatggtgttgcggctgttgctgttgctgcaaatgaagttggtggtggtggtggtacaactgctcatcctgtaacatctgttcgtgttgacgctggcgagtctgcatctgtcgttgtagctgctcttcttcaaaacgagaacggggagaagaaagagggagaatataagggagggagacgggcgctgagaaaatgaatcaactggactgagacgcac

SEQ ID NO: 4 - DNA, 280 bp, synthetic construct
  source 1..280: mol_type = other DNA
tgacgttggtggagattccgaagatattggcgctcttgtaaaatttgctgatgatgagactcatggtgttgcggctgttgctgttgctgcaaatgaagttggtggtggtggtggtggtacaactgctcatcctgtaacatctgttcgtgttgacgctggcgagtctgcatctgtcgttgtagctgctcttcttcaaaacgagaacggggagaagaaagagggagaatataagggagggagacgggcgctgagaaaatgaatcaactggactgagacgcac

SEQ ID NO: 5 - DNA, 38 bp, synthetic construct
  source 1..38: mol_type = other DNA
tgtaaaacgacggccagtgtgacacagcaactcggaga

SEQ ID NO: 6 - DNA, 20 bp, synthetic construct
  source 1..20: mol_type = other DNA
cgatgtcttacggcagcgta

SEQ ID NO: 7 - DNA, 219 bp, synthetic construct
  source 1..219: mol_type = other DNA
gtgacacagcaactcggagaactgttgtacaagtaaatgttacacgaagcacaacaggagaactatatatatataattatggactgacgtgaaaatgcaggcgattactagtcccgttaagaggttgttatgaaaggaggtaaggaataacttggagaaaacggaaggtccatcccgaccagagtgcacgcatcaataatacgctgccgtaagacatcg

SEQ ID NO: 8 - DNA, 221 bp, synthetic construct
  source 1..221: mol_type = other DNA
gtgacacagcaactcggagaactgttgtacaagtaaatgttacacgaagcacaacaggagaactatatatatatataattatggactgacgtgaaaatgcaggcgattactagtcccgttaagaggttgttatgaaaggaggtaaggaataacttggagaaaacggaaggtccatcccgaccagagtgcacgcatcaataatacgctgccgtaagacatcg

SEQ ID NO: 9 - DNA, 38 bp, synthetic construct
  source 1..38: mol_type = other DNA
tgtaaaacgacggccagtcggtcgagcttcaggagttt

SEQ ID NO: 10 - DNA, 20 bp, synthetic construct
  source 1..20: mol_type = other DNA
accacgtcagccagaagatg

SEQ ID NO: 11 - DNA, 239 bp, synthetic construct
  source 1..239: mol_type = other DNA
cggtcgagcttcaggagttttgggttcgacgacctcttcttcttccacaacatcagtcttggattccacatcattgtgcaggaaggatcgttccttcttcctcttctacgcttccttcctcagaataattggtgtcctcgtcttcatcatcatcatcatcatcgagaatgacagaatgacgacctttaaatgagtaaccatcgaatacgtcaacggtatcatcttctggctgacgtggt

SEQ ID NO: 12 - DNA, 242 bp, synthetic construct
  source 1..242: mol_type = other DNA
cggtcgagcttcaggagttttgggttcgacgacctcttcttcttccacaacatcagtcttggattccacatcattgtgcaggaaggatcgttccttcttcctcttctacgcttccttcctcagaataattggtgtcctcgtcttcatcatcatcatcatcatcatcgagaatgacagaatgacgacctttaaatgagtaaccatcgaatacgtcaacggtatcatcttctggctgacgtggt

SEQ ID NO: 13 - DNA, 245 bp, synthetic construct
  source 1..245: mol_type = other DNA
cggtcgagcttcaggagttttgggttcgacgacctcttcttcttccacaacatcagtcttggattccacatcattgtgcaggaaggatcgttccttcttcctcttctacgcttccttcctcagaataattggtgtcctcgtcttcatcatcatcatcatcatcatcatcgagaatgacagaatgacgacctttaaatgagtaaccatcgaatacgtcaacggtatcatcttctggctgacgtggt